(19) United States
(12) Patent Application Publication
(10) Pub. No.: US 2008/0004620 A1
(43) Pub. Date: Jan. 3, 2008

(54) Device for resection of tissue

(75) Inventors: Michael D. Laufer, Menlo Park, CA (US); Jeffrey J. Christian, Morgan Hill, CA (US)

Correspondence Address: THELEN REID BROWN RAYSMAN & STEINER LLP, P. O. BOX 640640, SAN JOSE, CA, 95164-0640, US

(73) Assignee: Cithara Endoscopy, Inc., a California Corporation

(21) Appl. No.: 11/711,498

(22) Filed: Feb. 26, 2007

Related U.S. Application Data
(63) Continuation-in-part of application No. 10/649,047, filed on Aug. 26, 2003, now abandoned.
(60) Provisional application No. 60/435,986, filed on Dec. 20, 2002.

(51) Int. Cl. A61B 18/18

(52) U.S. Cl. 606/46

(57) ABSTRACT

The present invention provides for an apparatus and method to excise a tissue sample having a conducting element configured to receive power, an insulating holder coupled to said conducting element, and a connector coupled to said insulating holder for connection to a medical device.

### CROSS-REFERENCE TO RELATED APPLICATIONS

This application is a continuation in part of and claims priority under 35 U.S.C. §120 to, and incorporates by reference herein in its entirety, co-pending U.S. patent application Ser. No. 10/649,047 filed Aug. 26, 2003 by inventors Michael D. Laufer et al. entitled “Device for Resection of Tissue”. This application also claims the benefit of Provisional Patent Application Ser. No. 60/435,986, filed Dec. 20, 2002, by inventor Michael D. Laufer, M.D., entitled “NOVEL DEVICE FOR RESECTION OF TISSUE.”.

### FIELD OF THE INVENTION

The present invention relates to medical devices. More particularly, the present invention relates to a medical device for the excision of tissue.

### BACKGROUND OF THE INVENTION

Almost everyone experiences a little acid reflux, particularly after meals. Acid reflux irritates the walls of the esophagus, inducing a secondary peristaltic contraction of the smooth muscle, and may produce the discomfort or pain known as heartburn. Many people experience heartburn at least once a month and most episodes of acid reflux are asymptomatic. However, patients with a condition known as chronic gastroesophageal reflux disease (“GERD”), suffer from severe heartburn.

After a meal, the lower esophageal sphincter (“LES”) usually remains closed. When it relaxes, it may allow acid, partially digested foodstuff, and the like to reflux into the esophagus. Patients with GERD experience an increased number of transient LES relaxations, which are the dominant cause of reflux episodes. As the number of transient LES relaxations increases, the frequency of reflux episodes increases, thereby increasing the cumulative amount of time gastric acid spends in the esophagus. GERD symptoms are present weekly in nearly 20% of adults and daily in about 10% of adults.

Another factor that increases esophageal acid exposure time in patients with GERD is ineffective esophageal clearance. Although peristalsis (the movement of the esophagus, induced by swallowing, in which waves of alternate circular contraction and relaxation propel the contents onward) occurs, esophageal clearance is ineffective because of decreased amplitude of secondary peristaltic waves.

These gastric acids and other refluxing materials can cause irritation to the lower esophagus that in turn results in changes to the tissue. These changes, called metaplasia, are seen micro and macroscopically and if left unchecked can result in cancer of the esophagus. The pre-cancerous condition of metaplasia in the esophagus is known as Barrett's esophagus (“B.E.”). B.E. may also result from the abnormal tissue repair in the setting of chronic GERD.

The only reliable way to diagnose B.E. is for a patient to undergo yearly endoscopy and biopsy to detect “gastric- or intestinal-appearing mucosa.” B.E. is found in 12% of patients undergoing endoscopy for GERD. Of that percentage, the risk of esophageal cancer (“EC”) is 50 to 100 times higher than other people who do not have B.E. The incidence of EC has increased at a rate faster than any other cancer. In fact, EC is the eighth most common cancer in the world.

There are no drugs or surgery that produce consistent regression of B.E. B.E. is currently treated by repeated frequent biopsies and cutting and removing the affected section of the esophagus. If cancer is detected in the biopsies, the stomach is pulled up into the chest to connect with the shorter remaining stump of esophagus connected to the mouth. This procedure has serious consequences and disadvantages for patients, may need to be performed several times in a patient's lifetime, and is quite costly.

Thus, there is a need for an apparatus and method to excise affected tissue without having a patient undergo a painful, complicated, risky, and difficult surgery. Moreover, there is a need for an apparatus and method that can resect affected tissue from a body part, such as an esophagus, while leaving the structural elements of the body part intact.

### BRIEF DESCRIPTION OF THE INVENTION

The present invention provides for an apparatus and method to excise a tissue sample having a conducting element configured to receive power, an insulating holder coupled to said conducting element, and a connector coupled to said insulating holder for connection to a medical device.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1A is an illustration of a resection device in accordance with one embodiment of the present invention.

FIG. 1B is an illustration of the conducting element of the device of FIG. 1A.

FIG. 2 illustrates the resection device removably attached to an endoscope.

FIG. 3 is an illustration of an example showing removable attachment of the resection device connected to an endoscope in accordance with one embodiment of the present invention.

FIG. 3a is an illustration of the spring tension device of the present embodiment.

FIG. 3b is an alternative to the spring tension device of the present embodiment.

FIG. 3c is another alternative to the spring tension device of the present embodiment.

FIG. 4 is an illustration of the resection device in an esophagus.

FIG. 5 is a block diagram illustrating a method of the present invention.

FIGS. 6-9 illustrate steps for using the present embodiment.

FIG. 10 illustrates an alternative embodiment of the present invention.

### DETAILED DESCRIPTION

Embodiments of the present invention are described herein in the context of a device for resection of tissue. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

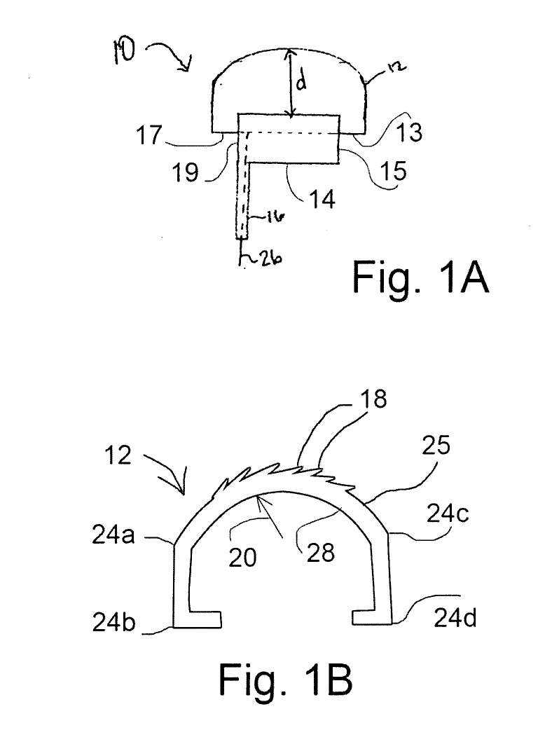

According to embodiments of the present invention, an apparatus and method to resect affected tissue from a body part, such as an esophagus, while leaving the structural elements of the body part intact is disclosed. FIG. 1A is an illustration of a resection device in accordance with one embodiment of the present invention. The resection device, generally numbered as 10, has a conducting element 12 mounted to an insulating holder 14. The conducting element 12 may be mounted to the insulating holder 14 with epoxy or any other similar material. The insulating holder 14 is substantially cylindrical, and a first section 13 of the conducting element 12 is connected to a first side 15 of the insulating holder 14 while a second section 17 of the conducting element 12 is connected to a second side 19 of the insulating holder 14. The conducting element 12 may be made of any conducting material and the insulating holder 14 may be made of any heat-resistant and electrically insulating material. In one embodiment of the present invention, the conducting element 12 is a wire made of tungsten and the insulating holder 14 may be made of ceramic. In another embodiment, the insulating holder 14 may be made of injection molded plastic. The distance d between the conducting element 12 and the insulating holder 14 determines the maximum depth of tissue cut. The actual depth of tissue cutting is determined by factors including the power setting and the tissue impedance.

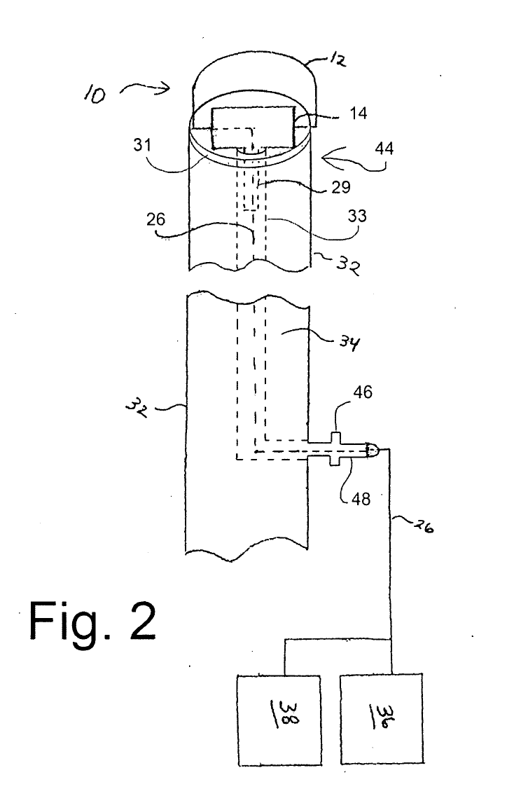

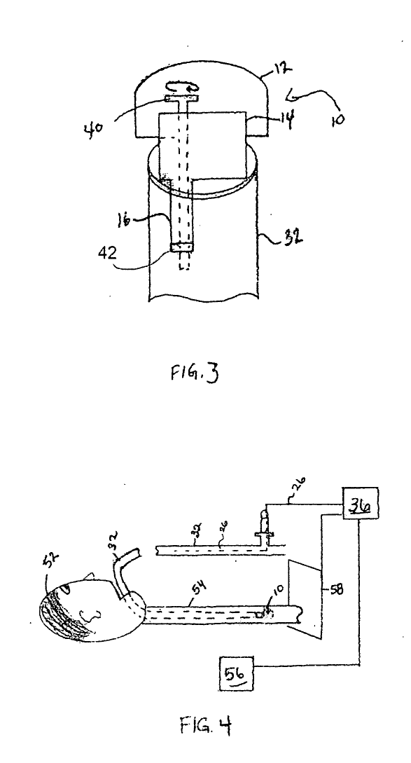

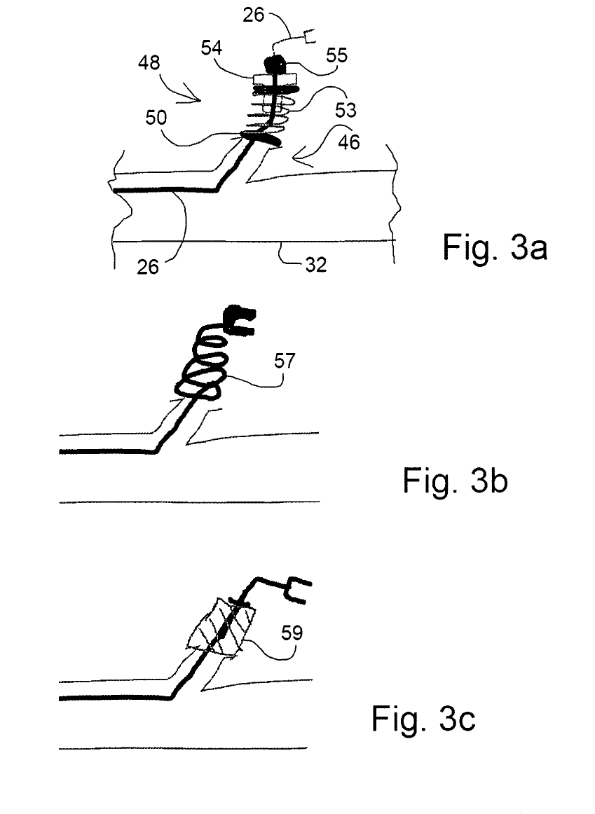

A connector 16 may be fixedly attached to the insulating holder 14 to connect the resection device 10 to a medical instrument such as an endoscope. The connector 16 is fixedly attached to one end of the insulating holder 14. However, as illustrated in FIGS. 2 and 3, the connector 29 may be fixedly attached to the center of the insulating holder 14 or other position. Thus, the position of the connector 16 is not intended to be limiting.

FIG. 1B is an illustration of the conducting element. The conducting element 12 is formed with many microfractures 18 along the top 22 of the conducting element 12. The microfractures 18 serve as current density concentration points to limit the plasma formed when the resection device 10 is activated to only those microfracture areas. The plasma acts to facilitate hemostasis of blood vessels and to separate the affected tissue from its tissue bed, thereby having the ability to cut strips of mucosa as further discussed below.

In the case where the conducting element 12 is formed from a tungsten wire, the microfractures 18 may be formed by bending the conducting element 12 along an arc 20 having a radius of less than about 5 cm. The conducting element 12 should not be pre-heated or annealed. In one embodiment, the conducting element 12 is bent at room temperature. Thus microfractures 18 in the form of microscopic “hairs” are formed on the surface of the tungsten wire. The corners 24a, 24b, 24c, 24d of the conducting element 12 may be bent to an angle of up to 90° to facilitate connection to the insulating holder 14. The microfractures 18 can also be made by abrading the wire with a diamond file of the appropriate grit size to create fractures of the desired size.

It should be understood that the microfractures 18 are located only on one side of the conducting element 12 while on the opposite side of the conducting element 12 the surface is relatively round and smooth and devoid of microfractures or “hairs”. In FIG. 1B the surface with microfractures is labeled 26 and is located on the longer side of the arc 20 while the surface with no substantial microfractures is labeled 28 and is located on the shorter side of the arc 20.

FIG. 2 illustrates the resection device removably attached to an endoscope. The resection device 10 may be removably attached to an optical endoscope 32 such as a fiber optic, charge coupled device, or any other similar endoscope. The connector 29 may be removably affixed to the distal plate 31 of the endoscope 32 by any means such as twisting, friction fit, screws, adhesive tape, or by any other similar means. The connector 29 extends through a hole in the distal plate 31 and into a working channel 33 which extends through the endoscope. The connector may be made of an elastomeric material.

FIG. 3 is an illustration of an example system to removably and flexibly attach the resection device connected to an endoscope in accordance with one embodiment of the present invention. FIG. 3 illustrates the use of a wing nut 40 to removably attach the resection device 10 to the endoscope 32. The wing nut 40 may be turned to bring in a pulling nut 42 closer to the wing nut 40. As a result, the connector 16 may bow or bulge. It is important that the connection within the working channel 33 continue to allow flexibility of the endoscope. Hence, a spring-loaded device 46 as shown is advantageous. Any fixed connector would effectively limit the flexible curvature of the endoscope because of its inability to lengthen as the working channel 33 is lengthened while bending the endoscope 32. Other embodiments that accomplish the same end include using a coil as at least a part of the conductor between the cutting end and the connector. Another embodiment includes a redundant fold of wire connected with an elastic material such that the fold can unfold to effectively lengthen the connector wire when the endoscope is bent.

Referring again to FIG. 2, the resection device 10 has an electrical connection fixedly attached to the conducting element 12 to provide the electrical energy from an energy source. The electrical connection may be formed by an electrical wire 26 inserted through a lumen within the working channel 33 of endoscope 32. In some cases a wire reinforcement member (not shown) may be located adjacent the first end 44 of the endoscope. The wire reinforcement member can include a screw-type connection for securely and releaseably connecting the wire at the connector 29 to the wire 26 from the spring tension device 48 so that the two wires will not be easily separated from each other when pulling force is applied to the wire 26 by the spring tension device 48. The electrical wire 26a may then exit an exit port 46 and be connected to a power source 36. In one embodiment, the power source 36 may supply radio frequency power.

A spring tension device 48 and friction tension device 50 may be positioned adjacent the exit port 46. With reference to FIG. 3a, the spring tension device 48 includes a helical spring 53 mounted surrounding the wire 26 distally of the friction tension device 50, a washer 54 mounted distally of the spring 53, and a retaining clip 55 fixedly mounted to the wire 26 distally of the washer. In use, the electrical wire 26 is pulled out through the friction tension device 50 until the resection device 10 is held adjacent the first end 44 while the endoscope 32 is in a straight position. The friction tension device 50 may then be actuated to secure the electrical wire 26 from withdrawing back into the endoscope 32 and out the first end 44. When the endoscope 32 is flexed, the spring 53 is compressed and the tension on the electrical wire 26 and resection device 10 may then be maintained. Conversely, when the endoscope resumes its straight position the spring 53 expands, thereby withdrawing the distal end of the wire 26 from the endoscope 32. This allows the resection device to be flexibly attached to the distal end of the endoscope with the resection device maintained in position by tension on the attached wire as it is pulled back by the spring tension device 48.

With reference now to FIGS. 3b and 3c, systems alternative to the spring tension system 48 are shown. In the embodiment of FIG. 3b the wire 26 is formed into a coil 57 which creates a spring. In the embodiment of FIG. 3c a cylinder of resilient material 59 is used to create spring forces.

With reference now to FIG. 4, the radio frequency power may be supplied in a bipolar fashion with the electrical wire 26 serving as one electrode. However, the power may be supplied in a monopolar fashion where the electrical wire 26 is one pole and the patient 52 is connected to the other circuit with a grounding plate 58.

The operation of the present embodiment can now be understood. With reference to FIG. 1A the distance d between the conducting element 12 and the insulating holder 14 determines the maximum depth of tissue cut. The actual depth of tissue cutting is determined by factors including the power setting and by the impedance of the tissue being cut. By reducing the power that is delivered to the wire 26, the cutting depth can be adjusted from surface only, or zero depth, to full thickness, limited only by the spacing d. Because the impedance of the tissue determines how the energy delivered interacts with the device, the impedance is also an important factor. As the tissue impedance increases, the energy delivered decreases (so long as the power is kept constant). Because the mucosa, or more superficial, tissue layer has less impedance than the submucosa (next deeper layer), the depth of cut can be limited to the mucosa only, by limiting the power to that which barely cuts at the impedance of the mucosa. When the wire hits the submucosa, the impedance increase causes the power to drop below the minimum cutting level, and the device cut is limited to the level of the mucosa.

It should be understood that the appropriate power level to cut just the mucosa but not the submucosa varies depending upon parameters such as the diameter of the wire forming the conducting element 12 and the characteristics of the micro fractures 18 of conducting element 12. After the appropriate power level has been experimentally determined for a particular conducting element a user can set that power level to be delivered by the power source 36 to enable the user to cut just the mucosa but not the submucosa.

The microfractures serve the additional function of providing a surface area of the conducting element 12 that is greater where the microfracture “hairs” exist (surface 25), and much less on the other side of the wire, where the surface is relatively round and smooth without the “hairs” (surface 28). As a result, the power density is greatest when only the tips of the “hairs” are in contact with the tissue, an event that occurs only when the cut into the mucosa is commenced. Once the hairs have become surrounded by tissue, which occurs after the cut was made into the mucosa, the wire begins to act as if it has no “hairs” and is a round wire. The entire surface of the wire now conducts into the tissue, and the power density is insufficient for the microfractures 18 to continue to provide plasma to cut, so long as the power is limited appropriately. The greatest concentration of cutting energy is now at the corners of the wire 24a, 24b, 24c, 24d where it is bent, because RF energy tends to focus at sharp corners. Therefore, the edges of the wire cut a strip of tissue while the long aspect of the wire primarily boils interstitial fluid which results in steam that aids in the dissection of the strip of tissue. This steam separates the cut mucosal tissue strip from the submucosal bed. The strips may then be removed and evaluated for cancerous cells. In contrast, current surgical ablation technologies do not allow for the removal of tissue for evaluation since the tissues are destroyed in situ without removing a sample of tissue. Likewise, devices that are designed to cut under water, such as those used for arthroscopic joint surgery, are incapable of this tissue-plane specific cutting and tissue-layer-specific depth discrimination.

As current is created by the electrical connection and the resection device 10 is moved between the layers of mucosa, steam is created. Tissue is dissected utilizing the steam that is created by the resistive heating of the conducting element 12 and/or the plasma field. It has been determined that the impedance of the mucosa and submucosa is different, possibly due to the greater percentage of moisture within the mucosa. This moisture difference results in a higher impedance in the submucosa and therefore less current flow to the submucosa. It is this impedance difference that allows the resection device to cut through the affected tissue and not damage the submucosa. However, a user will need to monitor and ensure that when the resection device is active and the tissue begins to desiccate, that the energy flow does not become re-concentrated at the “hairs” because the other parts of the wire are essentially insulated by non-conductive, dry tissue. This situation if not corrected by moving the device, could result in damage to the submucosa.

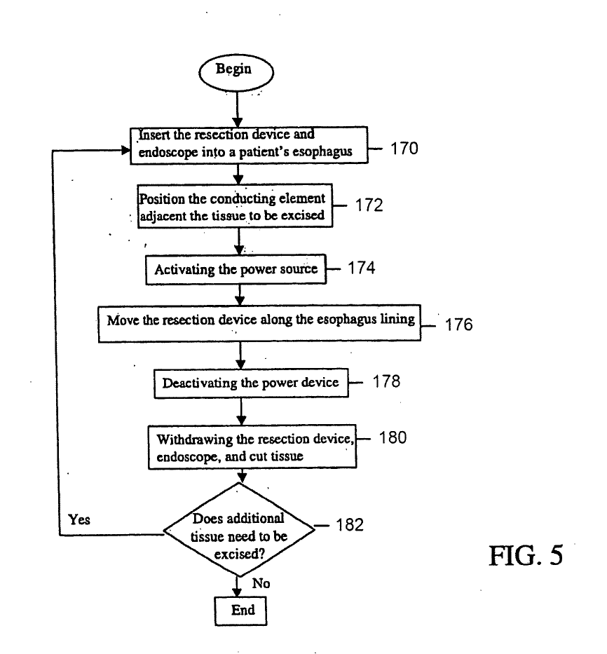

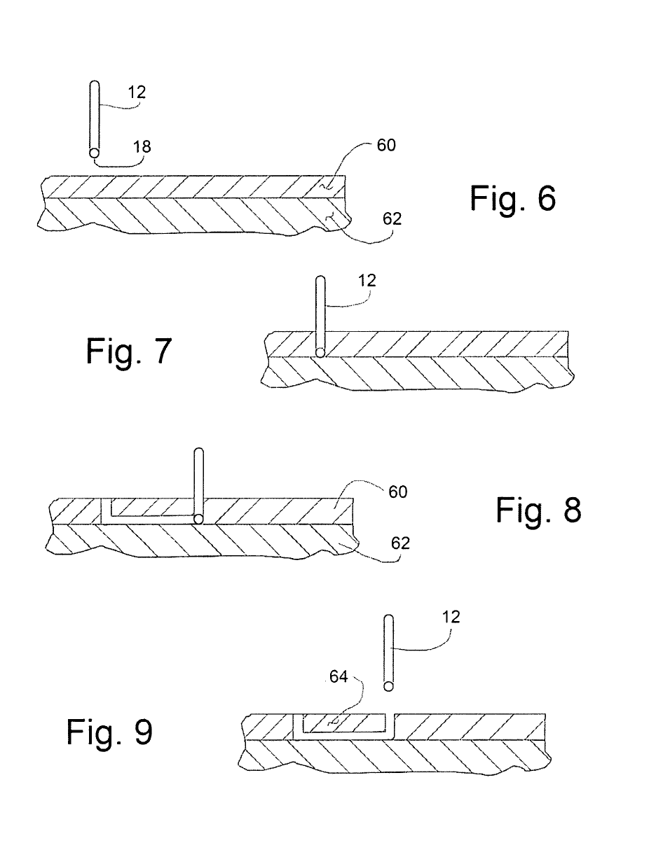

With reference now to FIGS. 5-9 the operation of the device is illustrated. FIG. 5 describes the stages of the resection process using the present embodiment. FIGS. 6-9 show steps of the resecting process in chronological sequence. It should be understood that FIGS. 6-9 are schematic, and e.g. only the conducting element 12, but not the holder 14 or the endoscope 32 are shown for simplicity.

In FIG. 6 the conducting element 12 is positioned near the patient's mucosal tissue 60, but resection has not yet begun. The physician then moves the conducting element 12 against the mucosal tissue 60, and plasma formed at the microfractures 18 enables the conducting element 12 to enter the mucosa 60. At this time, the power density is greatest when only the tips of the “hairs” are in contact with the tissue 60. Once the conducting element 12 has penetrated the mucosa and the hairs have become surrounded by tissue, which occurs after the cut was made into the mucosa, the wire begins to act as if it has no “hairs” and is a round wire. The entire surface of the wire now conducts into the tissue, and the operator controls the power supplied to conducting element so that the power density is insufficient for the microfractures 18 to continue to provide plasma to cut. The edges of the conducting element continue to cut the mucosa while the long aspect of the wire primarily boils interstitial fluid which results in steam that aids in tissue-layer separation.

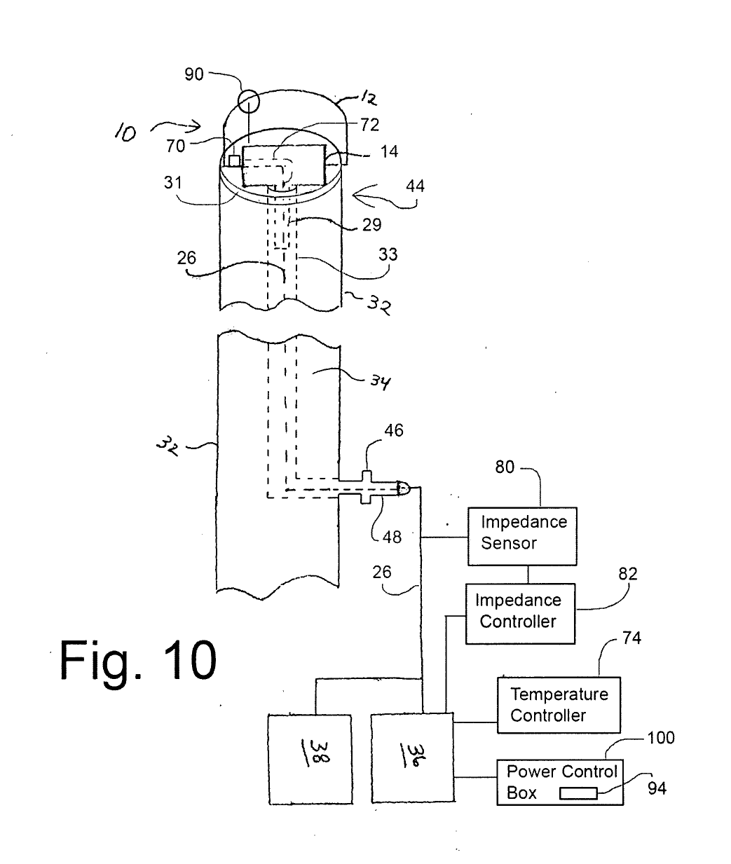

Because the mucosa 60 has less impedance than the submucosa 62, the depth of cut is limited to the mucosa 60 only, by limiting the power to that which barely cuts at the impedance of the mucosa. When the wire hits the submucosa 62 (FIG. 7), the impedance increase causes the power to drop below the minimum cutting level, and the device cut is limited to the level of the mucosa. The physician then moves the conducting element 12 toward the right in the mucosa to then allow resecting of a sample 64. (FIGS. 8-9)

It should be understood that, as long as steam is created by the conducting element 12, the temperature should be no greater than about 100° C. As the device only cuts when a circuit is present, as the fluid from the tissue is boiled away, the impedance rises and the cutting stops. The impedance rise can be sensed by the power source 36 and used to determine when the tissue has been desiccated as well as when the device has been kept immobile for too long. Impedance can be used to signal the operator or to control the rate of movement of the device directly. To move the cutter directly, a motorized mechanism may be included that effects such movement.

Alternative embodiments of the present invention may be used with other devices to enhance the performance of the resection device. A vibrating mechanism 38 may be removably attached to the resection device to increase the efficiency of separating the affected tissue from its tissue bed. The vibrating mechanism 38 may be a mechanical rotating vibrator or an ultrasonic vibrating crystal. As illustrated in FIG. 2, if a mechanical rotating vibrator is used, the mechanical rotating vibrator may be removably attached to the electrical wire 26. However, if an ultrasonic vibrating crystal is used, it may be integrated into the resection device 10 and coupled to the conducting element 12.

Various medical instruments may be removably attached or connected to the resection device to ensure accurate movement or incision of the resection device to prevent inadvertent perforation of non-affected tissue or body parts. Medical instruments that may be used to sense, monitor, and/or ensure movement of the resection device are temperature sensing devices, impedance sensing devices, direct motion sensing device, indirect motion sensing devices, mechanical pullers and/or pushers, and visualization as further described below.

Temperature sensing devices, such as a thermocouple or thermistor, may be attached to the conducting element 12. The temperature-sensing device may be programmed to reduce or stop the RF circuit when a certain temperature is reached. With reference to FIG. 10, a temperature sensor 70 is attached to the conducting element 12, and a wire 72 carries signals from the temperature sensor 70 to a temperature controller 74. The temperature controller 74 controls the power source 36. For the excision of the mucosa in the esophagus, it was determined that a temperature range of about 70° C. and 100° C. worked best. As discussed above, the temperature should not exceed about 100° C. to prevent injury or damage to deeper structures of the body part. As such, unlike in other devices where plasma arcing is used as the primary cutting mode, the plasma generation is intended to be very limited, primarily only to the initial cutting into the mucosa, but not thereafter while the strip is being mobilized, separated or cut.

As discussed above, the resection device should continually be moving if activated to prevent injury to deeper structures of the body part. Thus, an impedance-sensing device may also be used to ensure accurate movement of the resection device. The impedance-sensing device may detect the impedance of the RF circuit as current courses through the resection device. With reference to FIG. 10, an impedance sensor 80 is connected to the wire 26 and the impedance sensor 80 is in turn connected to an impedance controller 82 which is connected to control the power source 36. If the resection device is activated but not moved through the affected tissue, the impedance rises in a nearly linear fashion as the tissue is desiccated. In the alternative, if the impedance increases and decreases again cyclically, it is an indication that the RF circuit is not interrupted and the resection device is moving. The waveform may be analyzed by Fast Fourier Transform, with the frequency breakpoint shifting as the device is moved. If the device is not moving, the frequency breakpoint does not appreciably shift.

A wheel may also be attached to the resection device through the electrical wire to detect movement of the resection device. The wheel moves as the resection device is moving, and the wheel stops when the resection device stops moving. Should the wheel stop moving, it is an indication that the RF circuit is to be interrupted to prevent deeper tissue injury or perforation. Such feedback is provided to the RF generator controller. With reference to FIG. 10 the wheel 90 is mounted to the distal plate 31, and signals from the wheel are conveyed by wire 26 to the RF generator controller which is part of power source 36.

A mechanical pull or pusher device may also be used to detect movement of the resection device. The pull or pusher device may be attached to the endoscope. Power will flow to the resection device if tension is applied to the endoscope sufficient to push or pull the mechanical pull or pusher device. If tension is reduced to below a certain level, the RF may be made to stop thereby stopping cutting of the resection device.

A power control box 92 may also be positioned to control the power source. The power control box 92 provides for an additional safely measure by controlling the current or RF flow to the conducting element 12. In one embodiment, the power control box 92 provides greater power initially to start a cut through the mucosa. The power control box 92 then decreases the power to a certain maximum power determined by the user or to a level determined by power control algorithms to be the maximum safe power setting. The power control algorithms receive input from the temperature sensor 70, the impedance sensor 80 and the wheel 90 and can be implemented by computer system 94 contained in the power control box 92. One limiting factor in the algorithms would be plasma generation, which is not desired and would result in an immediate reduction of power. Another factor in the algorithms would be the ability to reach 100 degrees C. which is necessary to create dissecting steam. This power modulating function prevents inadvertent cutting or damage to the deeper tissue of the body part. In another embodiment, the power control box 92 may detect movement of the resection device to control the current or RF flow. In yet another embodiment, the power control box 92 may also limit the maximum current flow by dumping excess current or RF flow to ground if the user inadvertently sets the power to a dangerous level.

Embodiments of the present invention further provide for methods of resecting affected tissue and promoting hemostasis to blood vessels. As illustrated in FIGS. 4 and 5 and described below, these exemplary embodiments of the invention are described with reference to the resection of tissue in an esophagus. However, those of ordinary skill in the art will realize that the methods may be used to resection tissue in other parts of a patient's body. For example, similar methods may be used to remove sessile polyps or other tissue where the depth of cut in important to control.

FIG. 5 is a block diagram illustrating a method of the present invention. The endoscope 32 and resection device 10 are inserted into a patient's 52 esophagus 54 at step 170 using methods that are well known to those of ordinary skill in the art. The conducting element of the resection device 10 is positioned adjacent the tissue to be excised at step 172. The power source 36 may be activated at step 174 with the use of a foot pedal 56 to provide energy to the conducting element.

The amount of power required will vary depending on the tissue excised. However, for the excise of tissue in the esophagus, the power may be in a range of 20-300 Watts. It was determined that in this power range, non-affected tissue was not cut, but affected tissue was easy to cut into and to separate from its underlying support tissue.

As current is created by the power source to the resection device and as the resection device is moved between the affected and unaffected tissue, steam is created. The tissue is dissected utilizing the steam that is created by the resistive heating of the conducting element and/or the plasma field. It has been determined that the impedance of the mucosa and submucosa varies, possibly due to the greater percentage of moisture within the mucosa. This moisture difference results in a higher impedance in the submucosa and therefore less current flows to the submucosa. It is this impedance difference that allows the resection device to cut through the affected tissue and not damage the submucosa. Thus, the present invention provides for a safe way to excise tissue without cutting or damaging the deeper structures of the body part.

The resection device is moved along the esophagus lining at step 176 and as discussed above, should be continually moved to prevent damage or cutting of the esophagus. The user may visually watch the endoscopic images as the resection device is moved along the esophagus to ensure good contact between the conducting element and the esophagus lining. When the desired tissue is excised and cut, the power source is deactivated at step 178 by releasing the foot pedal 56. The excised tissue, endoscope, and resection device are then withdrawn from the patient at step 180. The excised tissue may be attached naturally to the conducting element and thus withdrawn when the endoscope is withdrawn. However, the tissue may also be extracted with graspers. If additional tissue needs to be excised, the method is repeated at step 182.

Embodiments of the present invention were tested in the esophagus of an animal. The resection device was attached to an RF electrosurgical generator and advanced into the esophagus. The RF energy was activated and a cut was made to separate the mucosa from the submucosa and deeper tissues of the esophagus. A clear and decisive separation of the mucosal tissue from the submucosa was obtained. Another similar excision was performed next to the initial excision and similar results were obtained. The esophagus was then excised and opened for analysis. It was clear that there were no perforations or burns to the esophagus and that the surface of the esophagus was completely denuded of mucosa.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. An apparatus to excise a tissue sample, comprising:
a conducting element configured to receive power from a power source, said conducting element having at least a first surface and a second surface;
an insulating holder coupled to said conducting element;
wherein said first surface has a plurality of fractures and said second surface has no substantial fractures.

2. An apparatus according to claim 1 wherein said conducting element is a wire having a first section, a second section, and a curved section located between said first section and said second section.

3. An apparatus according to claim 2 wherein said insulating holder has a first side and a second side, and said first section of said wire is connected to said first side of said connector, said second section of said wire is connected to said second side of said connector, and said curved section of said wire is spaced apart from said insulating holder.

4. An apparatus according to claim 3 wherein said second surface of said wire is located toward said insulating holder and said first surface of said wire is located away from said connector.

5. The apparatus of claim 1 wherein said conducting element is made of a conducting material.

6. The apparatus of claim 5 wherein said conducting material is tungsten wire.

7. The apparatus of claim 1 wherein said insulating holder is made of a heat-resistance and electrically insulating material.

8. The apparatus of claim 1 wherein said medical device comprises an optical endoscope.

9. The apparatus of claim 1 wherein said fractures are micro-fractures.

10. The apparatus of claim 1 wherein said fractures comprise a plurality of hairs.

11. The apparatus of claim 10 wherein said hairs are formed by bending said conducting element.

12. The apparatus of claim 1 wherein the power source comprises an electrical wire connected to said conducting element at a first end and a power supply at a second end.

13. The apparatus of claim 12 wherein said power supply is a radio frequency power supply.

14. The apparatus of claim 12 wherein said electrical wire is secured with a spring tension device and a friction tension device.

15. The apparatus of claim 1 further comprising a vibrating mechanism coupled to the conducting element.

16. The apparatus of claim 12 further comprising a vibrating mechanism coupled to said electrical wire.

17. The apparatus of claim 1 further comprising a temperature sensor coupled to said conducting element.

18. The apparatus of claim 1 further comprising an impedance sensor coupled to said conducting element.

19. The apparatus of claim 1 further comprising a mechanical puller coupled to said conducting element.

20. The apparatus of claim 1 further comprising a mechanical pusher coupled to said conducting element.

21. The apparatus of claim 1 further comprising a power control system coupled to said conducting element.

22. A method for excising a tissue sample from a body, comprising:
inserting a resection device into the body, the resection device having a conducting element configured to receive electrical power;
positioning the conducting element adjacent the tissue to be excised;
controllably applying power to the conducting element;
moving the resection device adjacent the tissue to be excised;
removing the power applied to the conducting element; and
withdrawing said resection device from the body.

23. The method of claim 22 further comprising sensing the temperature of said conducting element and controllably applying power to the conducting element based on the sensed temperature.

24. The method of claim 22 wherein power is controllably supplied so that the temperature of the conducting element is maintained in the range of about 70 degrees C. to about 100 degrees C.

25. The method of claim 22 further comprising sensing the impedance of said conducting element and controllably applying power to the conducting element based on the sensed impedance.

26. The method of claim 22 wherein said applying further comprises pushing a foot pedal.

27. The method of claim 22 wherein said removing further comprises releasing a foot pedal.

28. The method of claim 22 wherein said moving further comprises viewing the movements of said resection device.

29. The method of claim 22 wherein said withdrawing further comprises grasping said tissue sample with a grasper.

30. The method of claim 22 wherein said conducting element is made of a conducting material.

31. The method of claim 30 wherein said conducting material is a tungsten wire.

32. The method of claim 22 wherein said inserting further comprises connecting said resection device to a medical device.

33. The method of claim 32 wherein said medical device comprises an optical endoscope.

34. The method of claim 22 wherein said conducting element includes a plurality of micro-fractures.

35. The method of claim 22 further comprising vibrating said conducting element.

36. An apparatus for excising a tissue sample from a body, comprising:
means for inserting a resection device into the body, the resection device having a conducting element configured to receive electrical power;
power supply means for controllably supplying power to the conducting element;
means for moving the resection device along the tissue tract; and,
means for withdrawing said resection device from the body.

37. The apparatus of claim 36 further comprising means for sensing the temperature of said conducting element, wherein said power supply means is connected to receive signals from said temperature sensor and to control the power supplied to said conducting element based on the sensed temperature.

38. The apparatus of claim 36 further comprising means for sensing the impedance of said conducting element, wherein said power supply means is connected to receive signals from said means for sensing the impedance and to control the power supplied to said conducting element based on the sensed impedance.

39. The apparatus of claim 36 wherein said means for applying further comprises pushing a foot pedal.

40. The apparatus of claim 36 wherein said means for removing further comprises releasing a foot pedal.

41. The apparatus of claim 36 wherein said means for moving further comprises viewing the movements of said resection device.

42. The apparatus of claim 36 wherein said means for withdrawing further comprises grasping said tissue sample with a grasper.

43. The apparatus of claim 36 wherein said conducting element is made of a conducting material.

44. The apparatus of claim 43 wherein said conducting material is a tungsten wire.

45. The apparatus of claim 36 wherein said means for inserting further comprises connecting said resection device to a medical device.

46. The apparatus of claim 45 wherein said medical device comprises an optical endoscope.

47. The apparatus of claim 36 wherein said conducting element includes a plurality of micro-fractures.

48. The apparatus of claim 36 further comprising means for vibrating said conducting element.

49. An apparatus to excise a mucosa tissue layer from a submucosa tissue layer, comprising:
a conducting element configured to receive power; and,
power control means to limit the power supplied to said conducting element so that the power is sufficient to enable the conducting element to cut the mucosa but not sufficient to enable the conducting element to cut the submucosa.

50. An apparatus according to claim 49 wherein said power control means comprises an impedance sensor to sense the impedance of said conducting element

51. The apparatus of claim 49 wherein the mucosa tissue layer has a higher percentage of moisture than the submucosa tissue layer.

52. The apparatus of claim 49 further comprising:
an insulating holder coupled to said conducting element; and
a connector coupled to said insulating holder for connection to a medical device.

53. The apparatus of claim 49 wherein said conducting element is a tungsten wire.

54. The apparatus of claim 50 wherein said insulating holder is made of a heat-resistant and electrically insulating material.

55. The apparatus of claim 49 wherein said medical device comprises an optical endoscope.

56. The apparatus of claim 49 wherein said conducting element includes a plurality of
micro-fractures to produce a plasma field.

57. The apparatus of claim 49 wherein the power is a wire enforcement member coupled to said conducting element.

58. The apparatus of claim 49 wherein the power is an electrical wire connected to said conducting element at a first end and a power source at a second end.

59. The apparatus of claim 58 wherein said power source is a radio frequency power source.

60. The apparatus of claim 58 wherein said electrical wire is secured with a spring tension device and a friction tension device.

61. The apparatus of claim 49 further comprising a vibrating mechanism coupled to the conducting element.

62. The apparatus of claim 58 further comprising a vibrating mechanism coupled to said electrical wire.

63. The apparatus of claim 49 further comprising a temperature sensor coupled to said conducting element.

64. The apparatus of claim 49 further comprising an impedance sensor coupled to said conducting element.

65. The apparatus of claim 49 further comprising a mechanical puller coupled to said conducting element.

66. The apparatus of claim 49 further comprising a mechanical pusher coupled to said conducting element.

67. The apparatus of claim 49 further comprising a power control box coupled to said conducting element.
